(19) United States
(12) Patent Application Publication
(10) Pub. No.: US 2024/0058336 A1
(43) Pub. Date: Feb. 22, 2024

(54) DOSING OF FEDRATINIB

(71) Applicant: IMPACT BIOMEDICINES, INC., Summit, NJ (US)

(72) Inventors: Gopal KRISHNA, Scotch Plains, NJ (US); Ken OGASAWARA, Cambridge, MA (US)

(21) Appl. No.: 18/257,770

(22) PCT Filed: Dec. 15, 2021
(86) PCT No.: PCT/US2021/063563

§ 371 Date: Jun. 15, 2023

Related U.S. Application Data
(60) Provisional application No. 63/126,289, filed on Dec. 16, 2020.

(51) Int. Cl. A61K 31/506; A61P 35/02; A61K 31/51; A61K 31/4178; A61K 31/4196

(52) U.S. Cl. CPC A61K 31/506; A61P 35/02; A61K 31/51; A61K 31/4178; A61K 31/4196

(57) ABSTRACT

The present disclosure provides methods of treating myeloproliferative disorders in patients concurrently receiving a dual CYP2C19 and CYP3A4 inhibitor.

### CROSS-REFERENCE TO RELATED APPLICATIONS

This application claims the priority benefit of U.S. Provisional Application No. 63/126,289, filed Dec. 16, 2020, which is incorporated by reference herein in its entirety.

### FIELD

The present disclosure provides methods of treating myeloproliferative disorders in patients concurrently receiving a dual CYP2C19 and CYP3A4 inhibitor. Also provided are methods of increasing the safety and/or tolerability of a compound of formula (I), or a pharmaceutically acceptable salt and/or solvate thereof in patients concurrently receiving a dual CYP2C19 and CYP3A4 inhibitor.

### BACKGROUND

Myelofibrosis (“MF”) is a rare disease mainly affecting people of older age. MF is a BCR-ABL1-negative myeloproliferative neoplasm (“MPN”) that presents de novo (primary) or may be preceded by polycythemia vera (“PV”) or essential thrombocythemia (“ET”). Clinical features include progressive anemia, marked splenomegaly, constitutional symptoms (e.g., fatigue, night sweats, bone pain, pruritus, and cough), and weight loss. Median survival ranges from less than 2 years to over 15 years based on currently identified prognostic factors.

Fedratinib is an oral, potent small molecule inhibitor of wild type and mutationally activated Janus kinase 2 (JAK2) and FMS-like tyrosine (FLT) kinase 3 (FLT3) that is indicated for the treatment of adult patients with intermediate-2 or high-risk primary or secondary (post-polycythemia vera [PV] or post-essential thrombocythemia) myelofibrosis (MF). Fedratinib inhibits JAK2 wild-type (WT), activated mutant JAK2V617F, and FLT3 kinase, and is selective for JAK2 over JAK1, JAK3 and tyrosine kinase 2 (TYK2).

Fedratinib inhibits dysregulated JAK2 signaling that drives the pathogenesis of myeloproliferative neoplasms (MPNs), including MF and PV. In patient-derived cell lines expressing JAK2V617F and cells engineered to express WT JAK2 or JAK2V617F, fedratinib reduced phosphorylation of STATS/STATS, inhibited cell proliferation, and increased apoptosis. In mouse models of JAK2V617F-driven MPNs, fedratinib blocked phosphorylation of STATS/5, increased survival and improved disease-associated symptoms, including leukopenia, anemia, splenomegaly, and fibrosis.

Due to its complex pharmacokinetic profile, fedratinib's interaction with other drugs should be considered to anticipate potential drug-drug interactions and avoid any side effects that result from such interactions. For example, as concomitant administration of fedratinib with a dual CYP3A4 and CYP2C19 inhibitor has not yet been studied, the current fedratinib U.S. prescribing information advises that patients taking a dual CYP3A4 and CYP2C19 inhibitor should avoid taking fedratinib (INREBIC® Prescribing Information, Section 7.1).

### SUMMARY

In certain aspects, the present disclosure provides a method of treating a myeloproliferative disorder in a patient in need thereof, the method comprising administering to the patient an amount effective to treat a myeloproliferative disorder, of a compound of formula (I):

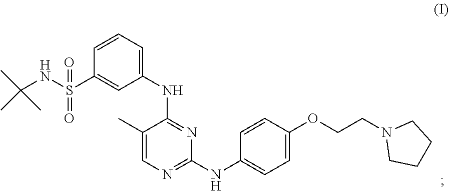 [SMILES: Cc1cnc(Nc2ccc(OCCN3CCCC3)cc2)nc1Nc1cccc(S(=O)(=O)NC(C)(C)C)c1]

or a pharmaceutically acceptable salt and/or a solvate thereof, wherein the patient is concurrently receiving a dual CYP2C19 and CYP3A4 inhibitor.

In some aspects, the solvate of the compound of formula (I), or the therapeutically acceptable salt thereof, is a hydrate. In some aspects, the pharmaceutically acceptable salt is a hydrochloride salt or a hydrate thereof. In some aspects, the dihydrochloride monohydrate of the compound of formula (I) is administered.

In certain aspects, the compound of formula (I), or the pharmaceutically acceptable salt and/or solvate thereof, is administered at a standard dose. In certain aspects, the compound of formula (I), or the pharmaceutically acceptable salt and/or solvate thereof, is administered at a dose less than a standard dose. In some aspects, a standard dose of the compound of formula (I) is about 400 mg/day.

In some aspects, the compound of formula (I), or the pharmaceutically acceptable salt and/or solvate thereof, is administered at a dose that is about ½ of a standard dose. In some aspects, the compound of formula (I), or the pharmaceutically acceptable salt and/or solvate thereof, is administered at a dose of about 200 mg/day.

In certain aspects, the compound of formula (I), or the pharmaceutically acceptable salt and/or solvate thereof, is administered at a dose that is about ¼ of a standard dose. In some aspects, the compound of formula (I), or the pharmaceutically acceptable salt and/or solvate thereof, is administered at a dose of about 100 mg/day.

In certain aspects of the present disclosure, the compound of formula (I), or the pharmaceutically acceptable salt and/or solvate thereof, is administered once daily.

In certain aspects, the compound of formula (I), or the pharmaceutically acceptable salt and/or solvate thereof, is administered at a dose that is about ⅛ a standard dose. In some aspects, the compound of formula (I), or the pharmaceutically acceptable salt and/or solvate thereof, is administered at a dose of about 50 mg/day. In certain aspects, the compound of formula (I), or the pharmaceutically acceptable salt and/or solvate thereof, is administered every other day. In some aspects, the compound of formula (I), or the pharmaceutically acceptable salt and/or solvate thereof, is administered at a dose of about 100 mg every other day.

In certain aspects of the present disclosure, the compound of formula (I), or the pharmaceutically acceptable salt and/or solvate thereof, is administered orally.

In certain aspects, the present disclosure provides a method of treating a myeloproliferative disorder in a patient receiving a dual CYP2C19 and CYP3A4 inhibitor, the method comprising: (a) administering to the patient an amount effective to treat a myeloproliferative disorder, of a compound of formula (I):

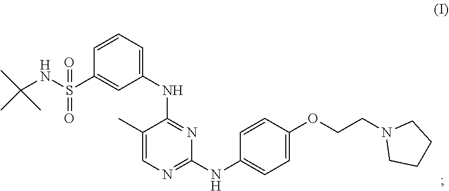 [SMILES: Cc1cnc(Nc2ccc(OCCN3CCCC3)cc2)nc1Nc1cccc(S(=O)(=O)NC(C)(C)C)c1]

or a pharmaceutically acceptable salt and/or a solvate thereof; (b) monitoring the patient for adverse reactions; and (c) in response to said monitoring, either maintaining or adjusting the dose of the compound of formula (I) or a pharmaceutically acceptable salt and/or a solvate thereof.

In some aspects, the dose of the compound of formula (I) or a pharmaceutically acceptable salt and/or a solvate thereof is maintained.

In some aspects, the dose of the compound of formula (I) or a pharmaceutically acceptable salt and/or a solvate thereof is adjusted.

In some aspects, the dose of the compound of formula (I) or a pharmaceutically acceptable salt and/or a solvate thereof is lowered.

In some aspects, the dual CYP2C19 and CYP3A4 inhibitor is selected from itraconazole, fluconazole, fluvoxamine, voriconazole, and combinations thereof. In some aspects, the dual CYP2C19 and CYP3A4 inhibitor is fluconazole.

In certain aspects of the present disclosure, the patient is administered a compound of formula (Ia):

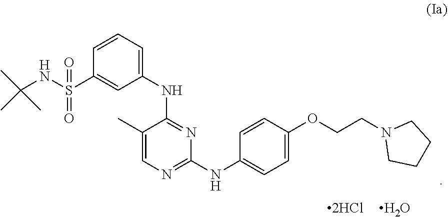 [SMILES: Cc1cnc(Nc2ccc(OCCN3CCCC3)cc2)nc1Nc1cccc(S(=O)(=O)NC(C)(C)C)c1 ; O]

In certain aspects, the present disclosure provides a method of treating a myeloproliferative disorder in a patient in need thereof, the method comprising administering to the patient an amount effective to treat a myeloproliferative disorder, of a compound of formula (I),

or a pharmaceutically acceptable salt and/or a solvate thereof, wherein the patient is concurrently receiving a dual CYP2C19 and CYP3A4 inhibitor, and wherein the myeloproliferative disorder is myelofibrosis. In some aspects, the myelofibrosis is primary myelofibrosis. In some aspects, the primary myelofibrosis is selected from intermediate risk primary myelofibrosis and high risk primary myelofibrosis. In some aspects, the myelofibrosis is secondary myelofibrosis. In some aspects, the myelofibrosis is post-essential thrombocythemia myelofibrosis. In certain aspects, the myelofibrosis is post-polycythemia vera myelofibrosis. In certain aspects the myeloproliferative disorder is acute myeloid leukemia (AML). In some aspects, the myeloproliferative disorder is polycythemia vera. In some aspects, the myeloproliferative disorder is essential thrombocythemia.

In certain aspects of the present disclosure, the method further comprises administering thiamine or a thiamine equivalent to the patient prior to, simultaneously with, or after the compound of formula (I), or a pharmaceutically acceptable salt and/or solvate thereof. In some aspects, the thiamine or thiamine equivalent is administered orally. In some aspects, the thiamine or thiamine equivalent is administered intravenously.

In certain aspects, the method further comprises administering a 5-HT3 receptor antagonist to the patient prior to, simultaneously with, or after the compound of formula (I), or a pharmaceutically acceptable salt and/or solvate thereof. In some aspects, the 5-HT3 receptor antagonist is ondasetron.

In certain aspects, the present disclosure provides a method of increasing the safety and/or tolerability of a compound of formula (I):

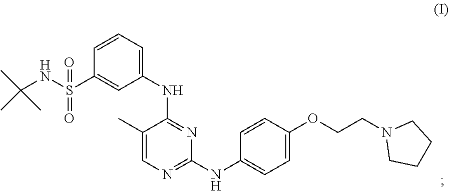 [SMILES: Cc1cnc(Nc2ccc(OCCN3CCCC3)cc2)nc1Nc1cccc(S(=O)(=O)NC(C)(C)C)c1]

or a pharmaceutically acceptable salt and/or a solvate thereof, the method comprising: (a) administering to a patient concurrently receiving a dual CYP2C19 and CYP3A4 inhibitor a dose of a compound of formula (I) or a pharmaceutically acceptable salt and/or solvate thereof (b) monitoring the patient for adverse reactions; and (c) in response to said monitoring, either maintaining or adjusting the dose of the compound of formula (I) or a pharmaceutically acceptable salt and/or a solvate thereof.

In some aspects, the dose of the compound of formula (I) or a pharmaceutically acceptable salt and/or a solvate thereof is maintained.

In some aspects, the dose of the compound of formula (I) or a pharmaceutically acceptable salt and/or a solvate thereof is adjusted.

In some aspects, the dose of the compound of formula (I) or a pharmaceutically acceptable salt and/or a solvate thereof is lowered.

In some aspects, the solvate is a hydrate. In some aspects, the pharmaceutically acceptable salt is a hydrochloride salt or a hydrate thereof. In some aspects, the dihydrochloride monohydrate of the compound of formula (I) is administered.

In some aspects, the compound of formula (I), or the pharmaceutically acceptable salt and/or solvate thereof, is administered at a standard dose.

In some aspects, the safety and/or tolerability is increased relative to that when the standard dose is administered to the patient. In some aspects, the solvate of the compound of formula (I) is a hydrate. In some aspects, the pharmaceutically acceptable salt is a hydrochloride salt or a hydrate thereof. In some aspects, the dihydrochloride monohydrate of the compound of formula (I) is administered.

In certain aspects, a standard dose of the compound of formula (I) is about 400 mg/day. In some aspects, the compound of formula (I), or the pharmaceutically acceptable salt and/or solvate thereof, is administered at a dose that is about ½ of a standard dose. In some aspects, the compound of formula (I), or the pharmaceutically acceptable salt and/or solvate thereof, is administered at a dose of about 200 mg/day. In some aspects, the compound of formula (I), or the pharmaceutically acceptable salt and/or solvate thereof, is administered at a dose that is about ¼ a standard dose. In some aspects, the compound of formula (I), or the pharmaceutically acceptable salt and/or solvate thereof, is administered at a dose of about 100 mg/day. In certain aspects, the compound of formula (I), or the pharmaceutically acceptable salt and/or solvate thereof, is administered once daily.

In some aspects, the compound of formula (I), or the pharmaceutically acceptable salt and/or solvate thereof, is administered at a dose that is about ⅛ a standard dose. In some aspects, the compound of formula (I), or the pharmaceutically acceptable salt and/or solvate thereof, is administered at a dose of about 50 mg/day. In some aspects, the compound of formula (I), or the pharmaceutically acceptable salt and/or solvate thereof, is administered every other day. In certain aspects, the compound of formula (I), or the pharmaceutically acceptable salt and/or solvate thereof, is administered at a dose of about 100 mg every other day.

In some aspects of the present disclosure, the method further comprises administering an increased dose of the compound of formula (I), or the pharmaceutically acceptable salt and/or solvate thereof, if the patient discontinues receiving the dual CYP2C19 and CYP3A4 inhibitor. In some aspects, the increased dose is less than the standard dose. In some aspects, the increased dose is the standard dose.

In some aspects of the present disclosure, the compound of formula (I), or the pharmaceutically acceptable salt and/or solvate thereof, is administered orally.

In certain aspects, the dual CYP2C19 and CYP3A4 inhibitor is selected from itraconazole, fluconazole, fluvoxamine, voriconazole, and combinations thereof. In some aspects, the dual CYP2C19 and CYP3A4 inhibitor is fluconazole.

In certain aspects, the patient is administered a compound of formula (Ia):

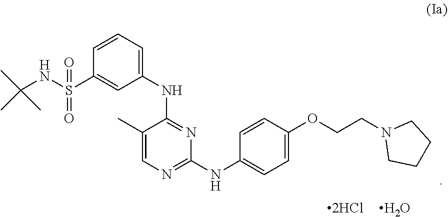 [SMILES: Cc1cnc(Nc2ccc(OCCN3CCCC3)cc2)nc1Nc1cccc(S(=O)(=O)NC(C)(C)C)c1 ; O]

In certain aspects, the method further comprises administering thiamine or a thiamine equivalent to the patient prior to, simultaneously with, or after the compound of formula (I), or a pharmaceutically acceptable salt and/or solvate thereof. In some aspects, the thiamine or thiamine equivalent is administered orally. In some aspects, the thiamine or thiamine equivalent is administered intravenously.

In some aspects, the method further comprises administering a 5-HT3 receptor antagonist to the patient prior to, simultaneously with, or after the compound of formula (I), or a pharmaceutically acceptable salt and/or solvate thereof. In certain aspects, the 5-HT3 receptor antagonist is ondasetron.

In certain aspects, administering the compound of formula (I), or the pharmaceutically acceptable salt and/or solvate thereof, treats a myeloproliferative disorder in the patient and reduces one or more conditions selected from anemia, thrombocytopenia, gastrointestinal toxicity, hepatic toxicity, amylase elevation, and lipase elevation. In some aspects, the myeloproliferative disorder is myelofibrosis. In some aspects, the myelofibrosis is primary myelofibrosis. In certain aspects, the primary myelofibrosis is selected from intermediate risk primary myelofibrosis and high risk primary myelofibrosis. In some aspects, the myelofibrosis is secondary myelofibrosis. In some aspects, the myelofibrosis is post-essential thrombocythemia myelofibrosis. In some aspects, the myelofibrosis is post-polycythemia vera myelofibrosis. In certain aspects, the myeloproliferative disorder is acute myeloid leukemia (AML). In some aspects, the myeloproliferative disorder is polycythemia vera. In some aspects, the myeloproliferative disorder is essential thrombocythemia.

In certain aspects, the increase in safety and/or tolerability comprises a reduction in toxicity and/or side effects. In some aspects, the improved safety and/or tolerability of the compound of formula (I), or the pharmaceutically acceptable salt and/or solvate thereof, comprises a reduction in one or more conditions selected from anemia, thrombocytopenia, gastrointestinal toxicity, hepatic toxicity, amylase elevation, and lipase elevation.

### DETAILED DESCRIPTION

The present disclosure provides methods of treating myeloproliferative disorders in patients concurrently receiving a dual CYP2C19 and CYP3A4 inhibitor. Also provided are methods of increasing the safety and/or tolerability of a compound of formula (I), or a pharmaceutically acceptable salt and/or solvate thereof in patients concurrently receiving a dual CYP2C19 and CYP3A4 inhibitor.

### Definitions

In order that the present description can be more readily understood, certain terms are first defined. Additional definitions are set forth throughout the detailed description.

It is to be noted that the term “a” or “an” entity refers to one or more of that entity. As such, the terms “a” (or “an”), “one or more,” and “at least one” can be used interchangeably herein. It is further noted that the claims can be drafted to exclude any optional element. As such, this statement is intended to serve as antecedent basis for use of such exclusive terminology as “solely,” “only” and the like in connection with the recitation of claim elements, or use of a negative limitation.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Units, prefixes, and symbols are denoted in their Système International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the disclosure. Thus, ranges recited herein are understood to be shorthand for all of the values within the range, inclusive of the recited endpoints. For example, a range of 1 to 10 is understood to include any number, combination of numbers, or sub-range from the group consisting of 1, 2, 3, 4, 5, 6, 7, 8, 9, and 10.

Where a value is explicitly recited, it is to be understood that values which are about the same quantity or amount as the recited value are also within the scope of the disclosure. Where a combination is disclosed, each subcombination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower). In some aspects of the formulations of the disclosure, the term “about” encompasses a deviation from the recited value of between 0.001% and 10%, inclusive of the endpoints. In some aspects, the term “about” encompasses an increase from the recited value of between 0.001% and 10%, inclusive of the endpoints. In some aspects, the term “about” encompasses a decrease from the recited value of between 0.001% and 10%, inclusive of the endpoints.

The terms “administration,” “administering,” and grammatical variants thereof refer to introducing a composition, such as fedratinib, of the present disclosure, into a subject via a pharmaceutically acceptable route. The introduction of a composition of the present disclosure into a subject is by any suitable route, including intratumorally, orally, pulmonarily, intranasally, parenterally (intravenously, intra-arterially, intramuscularly, intraperitoneally, or subcutaneously), rectally, intralymphatically, intrathecally, periocularly or topically. Administration includes self-administration and the administration by another. A suitable route of administration allows the composition or the agent to perform its intended function. For example, if a suitable route is intravenous, the composition can be administered by introducing the composition or agent into a vein of the subject.

The term “subject” refers to a human. The terms “subject” and “patient” are used interchangeably herein.

The term “effective amount” refers to an amount of an agent (e.g., fedratinib) that provides beneifical or desired therapeutic and/or prophylactic results. For prophylactic use, beneficial or desired results can include, for example, one or more results such as eliminating or reducing the risk, lessening the severity, or delaying the onset of the disease, including biochemical, histological, and/or behavorial symptoms of the disease, its complications, and intermediate pathological phenotypes presenting during development of the disease. For therapeutic use, beneificial or desired results can include, for example, one or more clinical results such as decreasing one or more symptoms and pathological conditions resulting from or associated with the disease, increasing the quality of life of those suffering from the disease, decreasing the dose of other medications required to treat the disease, enhancing the effect of other medication such as via targeting, delaying the progression of the disease, and/or prolonging survival. An effective amount can be, for example, an amount sufficient to accomplish prophylactic or therapeutic treatment either directly or indirectly. As it is understood in the clinical context, an effective amount of a drug, compound, or pharmaceutical composition may or may not be achieved in conjunction with another drug, compound, or pharmaceutical composition. Thus, an effective amount may be considered in the context of administering one or more therapeutic agents. An effective amount can be administered in one dosage or can be divided into multiple dosages, the total of such dosages being the effective amount. For example, an effective amount can be provided in two separate administrations over a period of time, that in aggregate, provide the effective amount of the formulation.

In certain aspects, the effective amount of fedratinib is the amount clinically proven to treat a myeloproliferative disorder such as myelofibrosis. The effective amount of fedratinib can have the effect in reducing one or more of splenomegaly, improving constitional symptoms (such as early satiety, fatigue, night sweats, cough, and pruritus), reducing leukocytosis, reducing thrombocytosis, decreasing JAK2V617F allele burden, reducing bone marrow fibrosis, and/or reducing bone marrow cellularity.

The terms “treat” or “treatment” refer to both therapeutic treatment and prophylactic or preventative measures, wherein the object is to prevent or slow down (lessen) an undesired physiological change or disorder. For purposes of this disclosure, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. Those in need of treatment include those already with the condition or disorder as well as those prone to have the condition or disorder or those in which the condition or disorder is to be prevented.

The term “side effect” refers to a secondary unwanted or unexpected event or reaction to a drug.

The term “toxicity” refers to the extent to which a drug is harmful or poisonous.

### Methods of Treatment

In certain aspects, the present disclosure provides methods of treating a myeloproliferative disorder in a patient in need thereof by administering a compound of formula (I), or a pharmaceutically acceptable salt and/or solvate thereof, wherein the patient is concurrently receiving a dual CYP2C19 and CYP3A4 inhibitor.

In some aspects, the patient receiving the compound of formula (I), or a pharmaceutically acceptable salt and/or solvate thereof, and a dual CYP2C19 and CYP3A4 inhibitor is being monitored. Such monitoring may include monitoring for side effects, safety and/or adverse events, for example, those related to fedratinib. In some aspects, monitoring for safey is as described in the prescribing information for INREBIC® (fedratinib). A physician may adjust the dose of the compound of formula (I), or a pharmaceutically acceptable salt and/or solvate thereof, for example, as recommended by the prescribing information for INREBIC® (fedratinib).

In some aspects, monitoring for safety is as recommended in the in the prescribing information INREBIC® (fedratinib):
Obtain the following blood tests prior to starting treatment with INREBIC, periodically during treatment, and as clinically indicated:
   Thiamine (Vitamin B1) level
   Complete blood count with platelets
   Creatinine and BUN
   Hepatic panel
   Amylase and lipase

In some aspects, the dose of the compound of formula (I), or a pharmaceutically acceptable salt and/or solvate thereof is adjusted as recommended in the prescribing information for concomitant use of INREBIC® (fedratinib) with strong CYP3A4 inhibitors:
Reduce INREBIC dose when administering with strong CYP3A4 inhibitors to 200 mg once daily.
In cases where co-administration with a strong CYP3A4 inhibitor is discontinued, INREBIC dosage should be increased to 300 mg once daily during the first two weeks after discontinuation of the CYP3A4 inhibitor, and then to 400 mg once daily thereafter as tolerated.

In some aspects, the dose of the compound of formula (I), or a pharmaceutically acceptable salt and/or solvate thereof is adjusted as recommended in the prescribing information INREBIC® (fedratinib) in case of adverse reactions:
Modify dose for hematologic and non-hematologic adverse reactions per Table A and Table B. Discontinue INREBIC® in patients unable to tolerate a dose of 200 mg daily. See Warnings and Precautions for other mitigating strategies in the prescribing information for INREBIC®.

TABLE A
Dose Modifications for Hematologic Adverse Reactions

| Hematologic Adverse Reactions | Dose Reduction |
|---|---|
| Grade 4 Thrombocytopenia or | Interrupt dose until resolved to Grade 2 |
| Grade 3 Thrombocytopenia with | or lower or baseline. Restart dose at |
| active bleeding | 100 mg daily below the last given dose. |
| Grade 4 Neutropenia | Interrupt dose until resolved to Grade 2 |
| | or lower or baseline. Restart dose at |
| | 100 mg daily below the last given dose. |

Consider dose reductions for patients who become transfusion-dependent during treatment with INREBIC®.

TABLE B
Dose Reductions for Non-hematologic Adverse Reactions

| Non-hematologic Adverse | |
|---|---|
| Reactions | Dose Reduction |
| Grade 3 or higher Nausea, | Interrupt dose until resolved to Grade 1 |
| Vomiting, or Diarrhea not | or lower or baseline. Restart dose at 100 |
| responding to supportive | mg daily below the last given dose. |
| measures within 48 hours | |
| Grade 3 or higher ALT, | Interrupt dose until resolved to Grade 1 |
| AST, or Bilirubin | or lower or baseline. Restart dose at 100 |
| | mg daily below the last given dose. |
| | Monitor ALT, AST, and bilirubin (total |
| | and direct) more frequently following |
| | the dose reduction. If re-occurrence of a |
| | Grade 3 or higher elevation, discontinue |
| | treatment with INREBIC ®. |
| Grade 3 or higher Other | Interrupt dose until resolved to Grade 1 |
| Non-hematologic | or lower or baseline. Restart dose at 100 |
| Toxicities | mg daily below the last given dose. |

In some aspects, the compound of formula (I), or the pharmaceutically acceptable salt and/or solvate thereof, can be administered orally, intravenously, intramuscularly, subcutaneously, peritoneally, intrathecally, intracranially, topically, vaginally, rectally, or any combination thereof. In certain aspects, the compound of formula (I), or the pharmaceutically acceptable salt and/or solvate thereof, can be administered orally. In some aspects, the compound of formula (I), or the pharmaceutically acceptable salt and/or solvate thereof, can be administered as a capsule. In some aspects, the compound of formula (I), or the pharmaceutically acceptable salt and/or solvate thereof, can be administered as a tablet.

In some aspects, the compound of formula (I), or the pharmaceutically acceptable salt and/or solvate thereof, can be administered at an effective amount for the method. In some embodiments, the compound of formula (I), or the pharmaceutically acceptable salt and/or solvate thereof, can be administered at a dose less than a standard dose. In some aspects, a standard dose of the compound of formula (I), or the pharmaceutically acceptable salt and/or solvate thereof, is about 400 mg/day. In some embodiments, the compound of formula (I), or the pharmaceutically acceptable salt and/or solvate thereof, can be administered at a dose that is about ⅛ a standard dose. In some embodiments, the compound of formula (I), or the pharmaceutically acceptable salt and/or solvate thereof, can be administered at a dose that is about ¼ of a standard dose. In some embodiments, the compound of formula (I), or the pharmaceutically acceptable salt and/or solvate thereof, can be administered at a dose that is about ½ of a standard dose.

In some aspects, the compound of formula (I), or the pharmaceutically acceptable salt and/or solvate thereof can be administered at from about 50 mg to about 700 mg, from about 75 mg to about 300 mg, or from about 90 mg to about 200 mg. In some aspects, the compound of formula (I), or the pharmaceutically acceptable salt and/or solvate thereof, can be administered at about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 550 mg, about 600 mg, about 650 mg, or about 700 mg, or an amount ranging from and to any of these values. In some aspects, the compound of formula (I), or the pharmaceutically acceptable salt and/or solvate thereof, can be administered at about 50 mg. In some aspects, the compound of formula (I), or the pharmaceutically acceptable salt and/or solvate thereof, can be administered at about 100 mg. In some aspects, the compound of formula (I), or the pharmaceutically acceptable salt and/or solvate thereof, can be administered at about 200 mg. In some aspects, the compound of formula (I), or the pharmaceutically acceptable salt and/or solvate thereof, can be administered at about 300 mg. In some aspects, the compound of formula (I), or the pharmaceutically acceptable salt and/or solvate thereof, can be administered at about 400 mg.

In some aspects, the compound of formula (I), or the pharmaceutically acceptable salt and/or solvate thereof, can be administered from 1 to 10 times a day. In some aspects, the compound of formula (I), or the pharmaceutically acceptable salt and/or solvate thereof, can be administered 1 to 5 times a day. In some aspects, the compound of formula (I), or the pharmaceutically acceptable salt and/or solvate thereof, can be administered 1, 2, 3, 4, 5, 6, 7, 8, 9, or 10 times a day. In some aspects, the compound of formula (I), or the pharmaceutically acceptable salt and/or solvate thereof, can be administered once a day. In some aspects, the compound of formula (I), or the pharmaceutically acceptable salt and/or solvate thereof, can be administered every other day. In some aspects, the compound of formula (I), or the pharmaceutically acceptable salt and/or solvate thereof, can be administered once every other day. In some aspects, the compound of formula (I), or the pharmaceutically acceptable salt and/or solvate thereof, can be administered every two days. In some aspects, the compound of formula (I), or the pharmaceutically acceptable salt and/or solvate thereof, can be administered once every two days.

In some aspects, the compound of formula (I), or the pharmaceutically acceptable salt and/or solvate thereof, can be administered at 50 mg once a day. In some aspects, the compound of formula (I), or the pharmaceutically acceptable salt and/or solvate thereof, can be administered at 100 mg once a day. In some aspects, the compound of formula (I), or the pharmaceutically acceptable salt and/or solvate thereof, can be administered at 100 mg once every other day. In some aspects, the compound of formula (I), or the pharmaceutically acceptable salt and/or solvate thereof, can be administered at 200 mg once a day. In some aspects, the compound of formula (I), or the pharmaceutically acceptable salt and/or solvate thereof, can be administered at 200 mg once every other a day. In some aspects, the compound of formula (I), or the pharmaceutically acceptable salt and/or solvate thereof, can be administered at 300 mg once a day. In some aspects, the compound of formula (I), or the pharmaceutically acceptable salt and/or solvate thereof, can be administered at 300 mg once every other day.

In some aspects, the compound of formula (Ia) can be administered at 50 mg once a day. In some aspects, the compound of formula (Ia) can be administered at 100 mg once a day. In some aspects, the compound of formula (Ia) can be administered at 100 mg once every other day. In some aspects, the compound of formula (Ia) can be administered at 200 mg once a day. In some aspects, the compound of formula (Ia) can be administered at 200 mg once every other a day. In some aspects, the compound of formula (Ia) can be administered at 300 mg once a day. In some aspects, the compound of formula (Ia) can be administered at 300 mg once every other day.

In some aspects, the myeloproliferative disorder treated by the compound of formula (I), or the pharmaceutically acceptables salt and/or solvate thereof, is myelofibrosis. In aspects, the myeloproliferative disorder can be myelofibrosis. In some aspects, the myelofibrosis can be primary myelofibrosis. In some aspects, the primary myelofibrosis can be Dynamic International Prognostic Scoring System (DIPSS) intermediate or high-risk primary myelofibrosis. In some aspects, the myelofibrosis can be secondary myelofibrosis. In some aspects, the myelofibrosis can be post-essential thrombocythemia myelofibrosis. In some aspects, the myelofibrosis can be post-polycythemia vera myelofibrosis. In some aspects, the myeloproliferative disorder can be polycythemia vera. In some aspects, the myeloproliferative disorder can be essential thrombocythemia. In some aspects, the myeloproliferative disorder can be acute myeloid leukemia (AML).

In certain aspects, the present disclosure provides methods of improving the safety and/or tolerability of a compound of formula (I), or a pharmaceutically acceptable salt and/or solvate thereof in a patient concurrently receiving a dual CYP2C19 and CYP3A4 inhibitor. In some aspects, improved safety and/or tolerability comprises a reduction in one or more conditions selected from anemia, thrombocytopenia, gastrointestinal toxicity, hepatic toxicity, amylase elevation, and lipase elevation.

In some aspects, the patient can be administered thiamine or a thiamine equivalent prior to, simultaneously with, or after the compound of formula (I), or the pharmaceutically acceptable salt and/or solvate thereof. In some aspects, the thiamine or thiamine equivalent can be administered orally. In some aspects, the thiamine or thiamine equivanelt can be administered intravenously. Methods of administering thiamine or thiamine equivalents with the compound of formula (I), or the pharmaceutically acceptable salt and/or solvate thereof, are disclosed in WO 2020/068755, which is incorporated by reference in its entirety.

In certain aspects, the patient can be administered a 5-HT3 receptor antagonist prior to, simultaneously with, or after the compound of formula (I), or a pharmaceutically acceptable salt and/or solvate thereof. In some aspects, the 5-HT3 receptor antagonis can be selected from ondansetron, granisetron, dolasetron, and palonosetron. In certain aspects, the 5-HT3 receptor antagonist is ondansetron.

In some aspects, the subject is less than 18 years old. In some aspects, the subject is 18 years or older. In certain aspects, the subject is between 18 years of age and 29 years of age, inclusive of the endpoints. In some aspects, the subject is between 30 years of age and 49 years of age, inclusive of the endpoints. In some aspects, the subject is between 50 years of age and 69 years of age, inclusive of the endpoints. In certain aspects, the subject is between 70 years of age and 100 years of age, inclusive of the endpoints.

In some aspects, the compound of formula (I), or the pharmaceutically acceptable salt and/or solvate thereof, can be administered to the subject prior to or after the administration of the dual CYP2C19 and CYP3A4 inhibitor. In other aspects, the compound of formula (I), or the pharmaceutically acceptable salt and/or solvate thereof, can be administered to the subject concurrently with the dual CYP2C19 and CYP3A4 inhibitor.

### Compound of Formula (I):

The present disclosure provides methods of treating a myeloproliferative disorder comprising administering to a patient in need thereof an effective amount of a compound of formula (I), or a pharmaceutically acceptable salt and/or solvate thereof, wherein the patient is concurrently receiving a dual CYP2C19 and CYP3A4 inhibitor. Also provided is a method of increasing the safety and/or tolerability of a compound of formula (I), or a pharmaceutically acceptable salt and/or solvate thereof in a patient concurrently receiving a dual CYP2C19 and CYP3A4 inhibitor.

“Pharmaceutically acceptable salts” are derived from inorganic or organic acids or bases. Examples of suitable acid addition salts include acetate, adipate, alginate, aspartate, benzoate, benzene sulfonate, bisulfate, butyrate, citrate, camphorate, camphor sulfonate, cyclopentanepropionate, digluconate, dodecylsulfate, ethanesulfonate, fumarate, lucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenyl-propionate, picrate, pivalate, propionate, succinate, tartrate, thiocyanate, tosylate and undecanoate.

Examples of suitable base addition salts include ammonium salts; alkali metal salts, such as sodium and potassium salts; alkaline earth metal salts, such as calcium and magnesium salts; salts with organic bases, such as dicyclohexylamine salts, N-methyl-D-glucamine; and salts with amino acids such as arginine, lysine, and the like.

For example, Berge lists the following FDA-approved commercially marketed salts: anions acetate, besylate (benzenesulfonate), benzoate, bicarbonate, bitartrate, bromide, calcium edetate (ethylenediaminetetraacetate), camsylate (camphorsulfonate), carbonate, chloride, citrate, dihydrochloride, edetate (ethylenediaminetetraacetate), edisylate (1,2-ethanedisulfonate), estolate (lauryl sulfate), esylate (ethanesulfonate), fumarate, gluceptate (glucoheptonate), gluconate, glutamate, glycollylarsanilate (glycollamidophenylarsonate), hexylresorcinate, hydrabamine (N,N′-di(dehydroabietyl)-ethylenediamine), hydrobromide, hydrochloride, hydroxynaphthoate, iodide, isethionate (2-hydroxyethanesulfonate), lactate, lactobionate, malate, maleate, mandelate, mesylate (methanesulfonate), methylbromide, methylnitrate, methylsulfate, mucate, napsylate (2-naphthalenesulfonate), nitrate, pamoate (embonate), pantothenate, phosphate/diphosphate, polygalacturonate, salicylate, stearate, subacetate, succinate, sulfate, tannate, tartrate, teoclate (8-chlorotheophyllinate) and triethiodide; organic cations benzathine (N,N′-dibenzylethylenediamine), chloroprocaine, choline, diethanolamine, ethylenediamine, meglumine (N-methylglucamine) and procaine; and metallic cations aluminum, calcium, lithium, magnesium, potassium, sodium and zinc.

Berge additionally lists the following non-FDA-approved commercially marketed (outside the United States) salts: anions adipate, alginate, aminosalicylate, anhydromethylenecitrate, arecoline, aspartate, bisulfate, butylbromide, camphorate, digluconate, dihydrobromide, disuccinate, glycerophosphate, hemisulfate, hydrofluoride, hydroiodide, methylenebis(salicylate), napadisylate (1,5-naphthalenedisulfonate), oxalate, pectinate, persulfate, phenylethylbarbiturate, picrate, propionate, thiocyanate, tosylate and undecanoate; organic cations benethamine (N-benzylphenethylamine), clemizole (1-p-chlorobenzyl-2-pyrrolildine-1′-ylmethylbenzimidazole), diethylamine, piperazine and tromethamine (tris(hydroxymethyl)aminomethane); and metallic cations barium and bismuth.

A “solvate” refers to a physical association of a compound of formula (I) with one or more solvent molecules, whether organic or inorganic. This physical association includes hydrogen bonding. In certain instances the solvate will be capable of isolation, for example when one or more solvent molecules are incorporated in the crystal lattice of the crystalline solid. Examples of solvates include, but are not limited to, hydrates, ethanolates, methanolates, isopropanolates, acetonitrile solvates, and ethyl acetate solvates. Methods of solvation are known in the art.

In some aspects, the compound of formula (I) can be present as a hydrate, which can be obtained, for example, by crystallization from a solvent or from aqueous solution. Any arbitrary number of solvate or water molecules can combine with the compound of formula (I) to form solvates and hydrates. Unless stated to the contrary, the disclosure includes all such possible solvates. Examples of solvates of the compound of formula (I) are disclosed in WO 2020/167845, which is hereby incorporated by reference.

In certain aspects, the compound of formula (I) is a compound of formula (Ia):

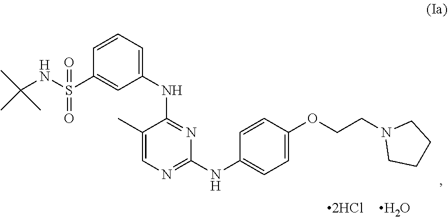 [SMILES: Cc1cnc(Nc2ccc(OCCN3CCCC3)cc2)nc1Nc1cccc(S(=O)(=O)NC(C)(C)C)c1 ; O]

known as fedratinib, which has the chemical name N-tert-butyl-3-[(5-methyl-2-{[4-(2-pyrrolidin-1-ylethoxy)phenyl]amino}pyrimidin-4-yl)amino]benzenesulfonamide dihydrochloride monohydrate, and has been previously described, e.g., in U.S. Pat. Nos. 7,528,143; 7,825,246; 8,138,199; and 10,391,094; and in PCT Application Publication Nos. WO 2020/167845 and WO 2020/068755, which are each hereby incorporated by reference in their entireties.

### Dual CYP2C19 and CYP3A4 Inhibitor

The present disclosure provides methods of treating a myeloproliferative disorder in a patient is concurrently receiving a dual CYP2C19 and CYP3A4 inhibitor. Also provided is a method of increasing the safety and/or tolerability of a compound of formula (I), or a pharmaceutically acceptable salt and/or solvate thereof in a patient concurrently receiving a dual CYP2C19 and CYP3A4 inhibitor. CYP2C19 (also known as cytochrome P450 2C19) and CYP3A4 (also known as cytochrome P450 3A4) are enzymes involved in drug metabolism. Without being bound by a particular theory, it is believed that the compound of formula (I) is largely metabolized by both of these enzymes. Thus, the exposure of the compound of formula (I) may be influenced by dual inhibitors of CYP2C19 and CYP3A4. Examples of compounds that inhibit CYP2C19 and CYP3A4 include, but are not limited to, itraconazole, fluconazole, fluvoxamine, voriconazole, and combinations thereof. In certain aspects of the present disclosure, the dual CYP2C19 and CYP3A4 inhibitor is selected from itraconazole, fluconazole, fluvoxamine, voriconazole, and combinations thereof. In some aspects, the dual CYP2C19 and CYP3A4 inhibitor is fluconazole.

### Compositions of Compound of Formula (I)

Pharmaceutical compositions comprising the compound of formula (I) can also comprise suitable carriers, excipients, and auxiliaries that may differ depending on the mode of administration.

The compound of formula (I) can be formulated for administration by a variety of means including orally, parenterally, by inhalation spray, topically, or rectally in formulations containing pharmaceutically acceptable carriers, adjuvants and vehicles. The term “parenteral” as used here includes subcutaneous, intravenous, intramuscular, and intraarterial injections with a variety of infusion techniques. Intraarterial and intravenous injection as used herein includes administration through catheters.

The compounds of the disclosure and the compositions of the disclosure can be formulated in accordance with the routine procedures adapted for desired administration route. Accordingly, the compositions of the disclosure can take such forms as suspensions, solutions or emulsions in oily or aqueous vehicles, and can contain formulatory agents such as suspending, stabilizing and/or dispersing agents. The compounds of the disclosure and the compositions of the disclosure can be formulated as a preparation suitable for implantation or injection. Thus, for example, the compound of formula (I) can be formulated with suitable polymeric or hydrophobic materials (e.g., as an emulsion in an acceptable oil) or ion exchange resins, or as sparingly soluble derivatives (e.g., as a sparingly soluble salt). The compounds of the disclosure and the compositions of the disclosure can be in powder form for constitution with a suitable vehicle, e.g., sterile pyrogen-free water, before use. Suitable formulations for each of these methods of administration can be found, for example, in Remington: The Science and Practice of Pharmacy, A Gennaro, ed., 20th edition, Lippincott, Williams & Wilkins, Philadelphia, PA.

In some aspects, the compositions of the disclosure are suitable for oral administration. These compositions can comprise solid, semisolid, gelmatrix or liquid dosage forms suitable for oral administration. As used herein, oral administration includes buccal, lingual, and sublingual administration. Suitable oral dosage forms include, without limitation, tablets, capsules, pills, troches, lozenges, pastilles, cachets, pellets, medicated chewing gum, granules, bulk powders, effervescent or non-effervescent powders or granules, solutions, emulsions, suspensions, solutions, wafers, sprinkles, elixirs, syrups or any combination thereof. In some aspects, compositions of the disclosure suitable for oral administration are in the form of a tablet or a capsule. In some aspects, the compound of the disclosure can be in the form of a capsule. In some aspects, capsules can be immediate release capsules.

The compositions of the disclosure can be in the form of compressed tablets, tablet triturates, chewable lozenges, rapidly dissolving tablets, multiple compressed tablets, or enteric-coating tablets, sugar-coated, or film-coated tablets. Enteric-coated tablets are compressed tablets coated with substances that resist the action of stomach acid but dissolve or disintegrate in the intestine, thus protecting the active ingredients from the acidic environment of the stomach. Enteric-coatings include, but are not limited to, fatty acids, fats, phenylsalicylate, waxes, shellac, ammoniated shellac, and cellulose acetate phthalates. Sugar-coated tablets are compressed tablets surrounded by a sugar coating, which can be beneficial in covering up objectionable tastes or odors and in protecting the tablets from oxidation. Film-coated tablets are compressed tablets that are covered with a thin layer or film of a water-soluble material. Film coatings include, but are not limited to, hydroxyethylcellulose, sodium carboxymethylcellulose, polyethylene glycol 4000, and cellulose acetate phthalate. A film coating can impart the same general characteristics as a sugar coating. Multiple compressed tablets are compressed tablets made by more than one compression cycle, including layered tablets, and press-coated or dry-coated tablets.

In some aspects, the compound of the disclosure can be in the form of a tablet. In some aspects, the compound of the disclosure can be in the form of a compressed tablet.

In some aspects, the compound of the disclosure can be in the form of a film-coated compressed tablet. In some aspects, the compositions of the disclosure can be in the form of film-coated compressed tablets.

In some aspects, the compositions of the disclosure can be prepared by fluid bed granulation of the compound of the disclosure with one or more pharmaceutically acceptable carriers, vehicles, and/or excipients. In some aspects, the compositions of the disclosure can be prepared by fluid bed granulation process and can provide a tablet formulation with good flowability, good compressibility, fast dissolution, good stability, and/or minimal to no cracking. In some aspects, the fluid bed granulation process can allow preparation of formulations having high drug loading, such as over 70% or over 75% of a compound of the disclosure.

In some aspects, the compositions of the disclosure can be in the form of soft or hard capsules, which can be made from gelatin, methylcellulose, starch, and/or calcium alginate. The hard gelatin capsule, also known as the dry-filled capsule (DFC), can comprise two sections, one slipping over the other, thus completely enclosing the active ingredient. The soft elastic capsule (SEC) is a soft, globular shell, such as a gelatin shell, which is plasticized by the addition of glycerin, sorbitol, or a similar polyol. In some aspects, tsoft gelatin shells can contain a preservative to prevent the growth of microorganisms. Suitable preservatives include, but are not limited to, those as described herein, including methyl- and propyl-parabens, sorbic acid, and combinations thereof. The liquid, semisolid, and solid dosage forms provided herein can be encapsulated in a capsule. Suitable liquid and semisolid dosage forms include, but are not limited to, solutions and suspensions in propylene carbonate, vegetable oils, triglycerides, and combinations thereof. Capsules containing such solutions can be prepared as described in U.S. Pat. Nos. 4,328,245; 4,409,239; and 4,410,545. The capsules can also be coated as known by those of skill in the art in order to modify or sustain dissolution of the active ingredient.

In some aspects, the compositions of the disclosure can be in liquid or semisolid dosage forms, including emulsions, solutions, suspensions, elixirs, and syrups. In some aspects, the emulsion can be a two-phase system, in which one liquid is dispersed in the form of small globules throughout another liquid, which can be oil-in-water or water-in-oil. Emulsions can include a pharmaceutically acceptable non-aqueous liquids or solvent, emulsifying agent, and preservative. Suspensions can include a pharmaceutically acceptable suspending agent and preservative. Aqueous alcoholic solutions can include a pharmaceutically acceptable acetal, such as a di-(lower alkyl)acetal of a lower alkyl aldehyde (the term “lower” means an alkyl having between 1 and 6 carbon atoms), e.g., acetaldehyde diethyl acetal; and a water-miscible solvent having one or more hydroxyl groups, such as propylene glycol and ethanol. Elixirs can be clear, sweetened, and hydroalcoholic solutions. Syrups can be concentrated aqueous solutions of a sugar, for example, sucrose, and can comprise a preservative. For a liquid dosage form, for example, a solution in a polyethylene glycol can be diluted with a sufficient quantity of a pharmaceutically acceptable liquid carrier, e.g., water, to be measured conveniently for administration.

In some aspects, the compositions of the disclosure for oral administration can be also provided in the forms of liposomes, micelles, microspheres, or nanosystems. Micellar dosage forms can be prepared as described in U.S. Pat. No. 6,350,458.

In some aspects, the compositions of the disclosure can be provided as non-effervescent or effervescent, granules and powders, to be reconstituted into a liquid dosage form. Pharmaceutically acceptable carriers and excipients used in the non-effervescent granules or powders can include, but are not limited to, diluents, sweeteners, wetting agents, and mixtures thereof. Pharmaceutically acceptable carriers and excipients used in the effervescent granules or powders can include, but are not limited to, organic acids, a source of carbon dioxide, and mixtures thereof.

Coloring and flavoring agents can be used in all of the above dosage forms. In addition, flavoring and sweetening agents can be especially useful in the formation of chewable tablets and lozenges.

In certain aspects, the compositions of the disclosure can be formulated as immediate or modified release dosage forms, including delayed-, extended, pulsed-, controlled, targeted-, and programmed-release forms.

The compositions of the disclosure can comprise another active ingredient that does not impair the composition's therapeutic or prophylactic efficacy and/or can comprise a substance that augments or supplements the composition's efficacy.

In certain aspects, the compound of formula (I), or the pharmaceutically acceptable salt and/or solvate thereof, can be administered orally. In some aspects, the compound of formula (I), or the pharmaceutically acceptable salt and/or solvate thereof, can be administered in a capsule. In some aspects, the compound of formula (I), or the pharmaceutically acceptable salt and/or solvate thereof, can be administered in a tablet.

In certain aspects, the compound of formula (I), or the pharmaceutically acceptable salt and/or solvate thereof, can be formulated as described in U.S. Pat. No. 10,391,094, which is incorporated herein by reference. In some aspects, the compositions of the disclosure comprising the compound of formula (I) or the pharmaceutically acceptable salt and/or solvate thereof can comprise from about 50 mg to about 700 mg, about 75 mg to about 400 mg, or about 90 mg to about 200 mg of the compound of formula (I) or the pharmaceutically acceptable salt and/or solvate thereof. In some aspects, the compositions of the disclosure can comprise a compound of formula (I) or a pharmaceutically acceptable salt and/or solvate thereof in an amount of about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 550 mg, about 600 mg, about 650 mg, or about 700 mg, or an amount ranging from and to any of these values. In some aspects, the compositions of the disclosure can comprise about 50 mg of a compound of formula (I) or a pharmaceutically acceptable salt and/or solvate thereof. In some aspects, the compositions of the disclosure can comprise about 100 mg of a compound of formula (I) or a pharmaceutically acceptable salt and/or solvate thereof. In some aspects, the compositions of the disclosure can comprise about 150 mg of a compound of formula (I) or a pharmaceutically acceptable salt and/or solvate thereof. In some aspects, the compositions of the disclosure can comprise about 50 mg of a compound of formula (Ia). In some aspects, the compositions of the disclosure can comprise about 100 mg of a compound of formula (Ia). In some aspects, the compositions of the disclosure can comprise about 150 mg of a compound of formula (Ia).

In certain aspects, compositions of the present disclosure can be administered from 1 to 10 times a day. In some aspects, the compositions of the present disclosure can be administered from 1 to 5 times a day. In some aspects, the compositions of the present disclosure can be administered 1, 2, 3, 4, 5, 6, 7, 8, 9, or 10 times a day. In some aspects, the compositions of the present disclosure can be administered once a day. In some aspects, the compositions of the present disclosure can be administered every other day. In some aspects, the compositions of the present disclosure can be administered once every other day. In some aspects, the compositions of the present disclosure can be administered every two days. In some aspects, the compositions of the present disclosure can be administered once every two days.

### EXAMPLES

### Example 1

### Phase I, Open-Label Study to Evaluate the Effect of a Dual CYP2C19 and CYP4A4 Inhibitor, Fluconazole, on the Pharmacokinetics of Fedratinib in Healthy Adult Subjects

A Phase 1, open-label study is being conducted to support all indications for which fedratinib is being developed. The primary objective is to evaluate the effect of multiple doses of a dual CYP2C19 and CYP3A4 inhibitor, fluconazole, on the pharmacokinetics (PK) of a single dose of fedratinib in healthy adult subjects. The secondary objective is to evaluate the safety and tolerability of a single dose of fedratinib when coadministered with or without fluconazole in healthy adult subjects. The exploratory objective of the study is to explore CYP and transporter biomarkers (eg, 4β-hydroxycholesterol), metabolites, and/or other transporter-related endogenous biomarkers.

Study Rationale and Purpose: Based on in vitro evaluations, fedratinib is metabolized by multiple CYPs, with the predominant contribution from CYP3A4, and with lesser contributions from CYP2C19 and flavin-containing monooxygenases (FMOs). Moreover, based on physiologically-based pharmacokinetic (PBPK) simulations, coadministration of a dual CYP2C19 and CYP3A4 inhibitor, fluconazole, is predicted to increase fedratinib exposure by approximately 4-fold.

This study is thus designed to test the effect of fluconazole (a dual CYP2C19 and CYP3A4 inhibitor) on the PK of fedratinib. Knowledge of these effects can be used to determine if dose adjustments should be considered when fedratinib is coadministered with drugs that are dualCYP2C19 and CYP3A4 inhibitors.

Rationale for the Study Design: This is an open-label study because the endpoints are objective parameters derived from measurements of plasma concentrations. The fixed-sequence, crossover design used in this study is typical for interaction studies where a relatively small number of subjects are required, because it removes intersubject variability from the comparison between treatments. Conducting the study in healthy subjects mitigates the potential confounding effects of the disease state and concomitant medications, and enrollment challenges with an MF patient population. Based on the effective t_{1/2} of fedratinib, the 17 days between doses of fedratinib is considered sufficient to prevent carryover to the second fedratinib dose.

Oral administration was chosen since this is the intended clinical route of administration of fedratinib. Based on the known PK of fedratinib, and accounting for potential reduction in clearance of fedratinib by inhibition of CYP2C19 and CYP3A4, the sample collection timing and duration of this study are considered adequate to achieve the study objectives. Fedratinib effective half-life is about 30 hours in healthy subjects, and by 168 hours postdose approximately 90% of the total fedratinib AUC is captured in healthy subjects. This supports the proposed fedratinib PK sampling interval in this study of up to 216 hours postdose, which takes into consideration likely changes in half-life.

Rationale for Dose, Schedule and Regimen Selection: The clinically recommended fedratinib dose is 400 mg QD. Further, single fedratinib doses of up to 680 mg have been tolerated by healthy subjects. The fedratinib dose in this study will be 100 mg so that fedratinib exposure, after the predicted ˜4-fold increase by a dual CYP2C19 and CYP3A4 inhibitor, remains in the therapeutic range and does not exceed that from a 680 mg dose in healthy subjects.

Ondansetron prophylaxis has been shown to reduce nausea and vomiting in a previous Phase 1 fedratinib study. To reduce the potential for fedratinib-related nausea and vomiting in this study, all subjects will receive an oral dose of 8 mg ondansetron approximately 1 hour before each fedratinib administration. A subsequent oral dose(s) of ondansetron may be given, as necessary, in accordance with the United States Package Insert (USPI).

As per the FDA Draft Guidance for Industry Clinical Drug Interaction Study Design, Data Analysis, and Clinical Implications (FDA, 2017), a single-dose study design is being used, since fedratinib PK in the clinically relevant dose range is linear with single-dose PK allowing prediction of multiple-dose PK. Clinically recommended doses of fluconazole (400 mg on the first day and then 200 mg QD thereafter) will be used during this study. Fluconazole will be orally administered for 8 days prior to coadministration with fedratinib in order to achieve stable dual inhibition of CYP2C19 and CYP3A4. Additional doses of fluconazole will be given after a single dose of fedratinib. This duration of fluconazole dosing was chosen based upon a previous successful study design evaluating effect of dual CYP2C19 and CYP3A4 inhibition on the PK of selumetinib (Dymond, 2017) and accounting for the long half-life of fedratinib.

Study Design: This is a study to evaluate the effect of a multiple doses of fluconazole on the PK, safety, and tolerability of a single dose of fedratinib in healthy adult subjects. The study will consist of a nonrandomized, fixed-sequence, open-label design. The subjects will participate as follows:
Screening phase
Treatment phase (includes baseline)
Follow-up telephone call

Subjects will be screened for eligibility. Subjects who meet all inclusion criteria and none of the exclusion criteria will return to the clinical site on Day −1 for protocol-specified assessments, and will be domiciled at the clinical site from Day −1 to Day 27. A single dose of fedratinib will be administered under fasted conditions on Day 1. Fedratinib will be washed out from Days 2 to 17. Subjects will receive fluconazole 400 mg on Day 10 and fluconazole 200 mg once daily (QD) on Days 11 to 23, inclusive. On Day 18, a single dose of fedratinib will be administered, under fasted conditions, with fluconazole.

Subjects Subjects will be discharged from the clinical site on Day 27 upon satisfactory safety review and completion of study-related procedures. Each subject will receive a follow-up telephone call 4 days (±2 days) after discharge. In the event a subject discontinues from the study for any reason, an early termination (ET) visit will be performed. Only the safety assessments scheduled for the day of discharge should be performed at the ET visit. Each discontinued subject should also receive a follow-up telephone call 4 days (±2 days) after completion of the ET visit.

During the study, blood samples will be collected at prespecified times for PK. Subject safety will be monitored throughout the study. The study will be conducted in compliance with the International Council on Harmonisation (ICH) of Technical Requirements for Registration of Pharmaceuticals for Human Use/Good Clinical Practice (GCP) and applicable regulatory requirements.

Approximately 16 healthy adult subjects will be enrolled into the study so that at least 12 subjects complete the study. The estimated duration from Screening through the follow-up telephone call is approximately 8 weeks. The End of Trial is defined as either the date of the last visit of the last subject to complete the post-treatment follow-up, or the date of receipt of the last data point from the last subject that is required for primary and/or secondary analysis, as prespecified in the protocol, whichever is the later date.

Treatment Administration and Schedule: On the evening before each dosing, all subjects will begin an overnight fast of at least 10 hours prior to dosing. No food or beverages (except water) will be allowed for at least 4 hours after dosing. Water is allowed as desired except for 1 hour before and 1 hour after each dosing.

Note: On Day 18, the duration of the overnight fast and postdose food/beverage (excluding water) restriction are relative to fluconazole dosing.

Each dose will be administered orally with approximately 240 mL noncarbonated, room temperature water. Dose modifications or interruptions are not permissible in this study. All subjects will receive the following oral doses of IP following an overnight fast in the fixed-sequence below:

Study Treatments: All subjects will receive the following oral doses of investigational product (IP) following an overnight fast in the fixed-sequence below:

Day 1: Single dose of 100 mg fedratinib (1×100-mg fedratinib capsule).

Days 10 to 23, inclusive: Single dose of 400 mg fluconazole on Day 10 (2×200-mg fluconazole tablets) and QD doses of 200 mg fluconazole on Days 11 to 23, inclusive (1×200-mg fluconazole tablet).

Day 18: Single dose of 100 mg fedratinib (1×100-mg fedratinib capsule) given with a dose of 200 mg fluconazole (1×200-mg fluconazole tablet).

All subjects will receive 8 mg oral ondansetron approximately 1 hour before each fedratinib administration to reduce the potential for fedratinib-related nausea and vomiting. A subsequent oral dose(s) of ondansetron may be given, as necessary, in accordance with the United States Package Insert (USPI).

Overview of Pharmacokinetic Assessments: Fedratinib plasma PK parameters will be calculated using noncompartmental methods. The following PK parameters will be estimated as allowed by the data:
Maximum observed plasma concentration (Cₘₐₓ)
Time to Cₘₐₓ (Tₘₐₓ)
Area under the plasma concentration-time curve (AUC) from time zero to the last quantifiable concentration (AUC₀₋ₜ)
AUC from time zero extrapolated to infinity (AUC_{0-∞}.)
Terminal elimination half-life (t_{1/2})
Apparent total plasma clearance when dosed orally (CL/F)
Apparent total volume of distribution when dosed orally (Vz/F), based on the terminal phase

All safety assessments will be tabulated and summarized as appropriate. Plasma concentrations and PK parameters will be listed and summarized using descriptive statistics.

To compare fedratinib PK parameters following single-dose administration in the presence and absence of QD doses of fluconazole, an analysis of variance model (ANOVA) with treatment as a fixed effect and subject as a random effect will be performed on the natural log-transformed Cₘₐₓ, AUC₀₋ₜ, and AUC_{0-∞}. The geometric means along with ratios of the geometric means (expressed as a percentage) and associated 90% confidence intervals (CIs) will be presented for the following PK parameter comparison:
fedratinib plus fluconazole (test) versus fedratinib alone (reference).

For Tₘₐₓ, Wilcoxon signed-rank test, Hodges-Lehmann estimate, and its 90% CI will be calculated for the median difference between treatments. Exploratory modeling analysis may be conducted to further evaluate the potential effect of DDI.

TABLE 1
Study Endpoints

| Endpoint | Name | Description | Timeframe |
|---|---|---|---|
| Primary | Pharmacokinetic | Cₘₐₓ and AUC for | Planned timepoints |
| | parameters | fedratinib. | throughout the study. |
| Secondary | Safety and tolerability | AEs, SAEs, vital sign | AEs: from the time the |
| | | measurements, clinical | ICF is signed until |
| | | laboratory safety tests | study completion and |
| | | (including hematology, | up to 30 days after |
| | | chemistry, and | the last dose of IP, |
| | | urinalysis), 12-lead | when made known to |
| | | ECGs, review of | the Investigator. |
| | | concomitant | SAEs: Same timeframe |
| | | medication and | as for AEs and at any |
| | | procedures | time thereafter that are |
| | | | suspected of being |
| | | | related to the IP will |
| | | | be recorded. |
| | | | All other safety |
| | | | assessments: as |
| | | | specified in the Table |
| | | | of Events. |
| Exploratory | CYP and transporter | Plasma levels of CYP | Any of the planned |
| | biomarkers, metabolites, | and transporter | PK sampling timepoints |
| | and/or other transporter | biomarkers (eg., (eg, | throughout the study. |
| | endogenous biomarkers | 4β-HC), metabolites, | |
| | | and/or other transporter | |
| | | endogenous biomarkers | |
| | | may be measured. | |

4β-HC = 4 beta-hydroxycholesterol; AE = adverse event; AUC = area under the plasma concentration-time curve; Cₘₐₓ = maximum observed plasma concentration; CYP = cytochrome P450; ECG = electrocardiogram; ICF = informed consent form; IP = investigational product;
PK = pharmacokinetic; SAE = serious adverse event.

Pharmacokinetics: Pharmacokinetic blood sampling should be performed at the nominal time(s) specified in this clinical protocol. Fedratinib plasma PK concentrations will be measured using a validated liquid chromatography tandem mass spectrometry assay.

All actual PK blood sample collection times will be recorded in the source documents and electronic case report form (eCRF). Explanation should be provided in the source documents and eCRF for any missed or mishandled samples and for any samples collected outside the time windows specified in Table 2:

TABLE 2
Pharmacokinetic Blood Collection Windows

| | Allowed Pharmacokinetic Window |
|---|---|
| Nominal Time Predose | Within 60 minutes prior to dosing |
| 0.5 to 4 hours postdose, inclusive | ±5 minutes |
| 6 to 12 hours postdose, inclusive | ±10 minutes |
| 24 hours and beyond | ±60 minutes |

Approximately 3 mL whole blood will be collected at the following times relative to fedratinib dosing on Day 1 and Day 18: predose and at 0.5, 1, 1.5, 2, 3, 4, 6, 8, 12, 24, 48, 72, 120, 168, and 216 hours postdose.

In addition to being used for measurement of fedratinib plasma concentrations, these samples may be used for exploratory analyses of CYP and transporter biomarkers (eg, 4β-hydroxycholesterol), metabolites, and/or other transporter endogenous biomarkers.

Pharmacogenetics: A pharmacogenetic (PG) blood sample (up to 10 mL) for potential analysis of deoxyribonucleic acid (DNA) related to drug metabolism and transport will be collected before dosing on Day 1.

For the PG sample, processing times and storage of the sample should be recorded in the source documents. Specific details regarding the collection, processing, storage, and shipment of PG samples are provided separately (eg, via stand-alone Lab Manual or equivalent).

### Study Population Definitions

All subjects who receive at least one dose of IP will be included in the safety population. The safety population will be used in safety analyses.

All subjects who receive at least one dose of IP and have at least one measurable concentration datum will be included in the PK population. The PK population will be used in PK analyses.

For subjects who withdraw or are discontinued from the study, all available PK and safety data (including follow-up) will be listed and summarized to the point of withdrawal/discontinuation. The reason for withdrawal/discontinuation will be included in the final study report.

### Sample Size and Power Considerations

Approximately 16 subjects will be enrolled into the study so that at least 12 subjects complete the study. Discontinued subjects may be replaced at the discretion of the Investigator and Sponsor's Medical Monitor.

The precision in the comparison of PK parameters are calculated for different estimates of intrasubject standard deviation (SD) on the natural log scale and sample size (Table 3). The precision represents the width of the 90% CI of the geometric mean ratios on the original scale.

Based on results from earlier studies, the estimated intrasubject SD was 0.191 for Cₘₐₓ. Twelve subjects will provide adequate precision.

TABLE 3
Precision and Intrasubject Standard Deviation

| | Precision | | | |
|---|---|---|---|---|
| Sample | Intrasubject | Intrasubject | Intrasubject | Intrasubject |
| Size | SD = 0.2 | SD = 0.3 | SD = 0.4 | SD = 0.5 |
| n = 12 | 15.8% | 24.6% | 34.1% | 44.3% |
| n = 14 | 14.3% | 22.2% | 30.7% | 39.7% |
| n = 16 | 13.2% | 20.4% | 28.1% | 36.3% |

n = number of subjects; SD = standard deviation.

It is to be appreciated that the Detailed Description section, and not the Summary and Abstract sections, is intended to be used to interpret the claims. The Summary and Abstract sections may set forth one or more but not all exemplary aspects of the present disclosure as contemplated by the inventor(s), and thus, are not intended to limit the present disclosure and the appended claims in any way.

The present disclosure has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific aspects will so fully reveal the general nature of the disclosure that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

What is claimed is:

1. A method of treating a myeloproliferative disorder in a patient in need thereof, the method comprising administering to the patient an amount effective to treat a myeloproliferative disorder, of a compound of formula (I): 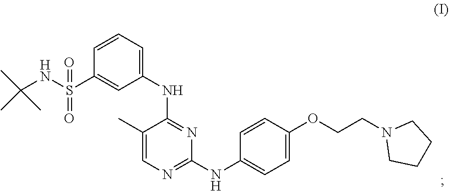 [SMILES: Cc1cnc(Nc2ccc(OCCN3CCCC3)cc2)nc1Nc1cccc(S(=O)(=O)NC(C)(C)C)c1]
or a pharmaceutically acceptable salt and/or a solvate thereof, wherein the patient is concurrently receiving a dual CYP2C19 and CYP3A4 inhibitor.

2. The method of claim 1, wherein the solvate is a hydrate.

3. The method of claim 1 or 2, wherein the pharmaceutically acceptable salt is a hydrochloride salt or a hydrate thereof.

4. The method of any one of claims 1 to 3, wherein the dihydrochloride monohydrate of the compound of formula (I) is administered.

5. The method of any one of claims 1 to 4, wherein the compound of formula (I), or the pharmaceutically acceptable salt and/or solvate thereof, is administered at a standard dose.

6. The method of any one of claims 1 to 4, wherein the compound of formula (I), or the pharmaceutically acceptable salt and/or solvate thereof, is administered at a dose less than a standard dose.

7. The method of claim 5 or 6, wherein the standard dose is about 400 mg/day.

8. The method of any one of claim 1 to 4, 6, or 7, wherein the compound of formula (I), or the pharmaceutically acceptable salt and/or solvate thereof, is administered at a dose that is about ½ of a standard dose.

9. The method of any one of claims 1 to 4 or 6 to 8, wherein the compound of formula (I), or the pharmaceutically acceptable salt and/or solvate thereof, is administered at a dose of about 200 mg/day.

10. The method of any one of claim 1-4, 6, or 7 wherein the compound of formula (I), or the pharmaceutically acceptable salt and/or solvate thereof, is administered at a dose that is about ¼ of a standard dose.

11. The method of any one of claim 1 to 4, 6, 7, or 10, wherein the compound of formula (I), or the pharmaceutically acceptable salt and/or solvate thereof, is administered at a dose of about 100 mg/day.

12. The method of any one of claims 1 to 11, wherein the compound of formula (I), or the pharmaceutically acceptable salt and/or solvate thereof, is administered once daily.

13. The method of any one of claim 1 to 4, 6, or 7, wherein the compound of formula (I), or the pharmaceutically acceptable salt and/or solvate thereof, is administered at a dose that is about ⅛ a standard dose.

14. The method of any one of claim 1 to 4, 6, 7, or 13, wherein the compound of formula (I), or the pharmaceutically acceptable salt and/or solvate thereof, is administered at a dose of about 50 mg/day.

15. The method of any one of claim 1-4, 6, 7, or 13, wherein the compound of formula (I), or the pharmaceutically acceptable salt and/or solvate thereof, is administered every other day.

16. The method of any one of claims 1 to 4, 6, 7, 13, and 15, wherein the compound of formula (I), or the pharmaceutically acceptable salt and/or solvate thereof, is administered at a dose of about 100 mg every other day.

17. The method of any one of claims 1 to 16, wherein the compound of formula (I), or the pharmaceutically acceptable salt and/or solvate thereof, is administered orally.

18. A method of treating a myeloproliferative disorder in a patient receiving a dual CYP2C19 and CYP3A4 inhibitor, the method comprising:
(a) administering to the patient an amount effective to treat a myeloproliferative disorder, of a compound of formula (I): 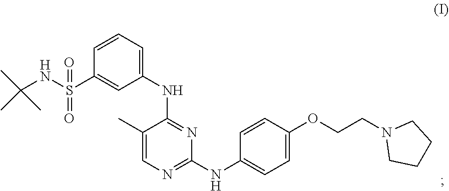 [SMILES: Cc1cnc(Nc2ccc(OCCN3CCCC3)cc2)nc1Nc1cccc(S(=O)(=O)NC(C)(C)C)c1]
or a pharmaceutically acceptable salt and/or a solvate thereof;
(b) monitoring the patient for adverse reactions; and
(c) in response to said monitoring, either maintaining or adjusting the dose of the compound of formula (I) or the pharmaceutically acceptable salt and/or solvate thereof.

19. The method of claim 18, wherein the dose of the compound of formula (I) or the pharmaceutically acceptable salt and/or solvate thereof is maintained.

20. The method of claim 18, wherein the dose of the compound of formula (I) or the pharmaceutically acceptable salt and/or solvate thereof is adjusted.

21. The method of claim 20, wherein the dose of the compound of formula (I) or the pharmaceutically acceptable salt and/or solvate thereof is lowered.

22. The method of any one of claims 1 to 21, wherein the dual CYP2C19 and CYP3A4 inhibitor is selected from itraconazole, fluconazole, fluvoxamine, voriconazole, and combinations thereof.

23. The method of any one of claims 1 to 22, wherein the dual CYP2C19 and CYP3A4 inhibitor is fluconazole.

24. The method of any one of claims 1 to 23, wherein the patient is administered a compound of formula (Ia):

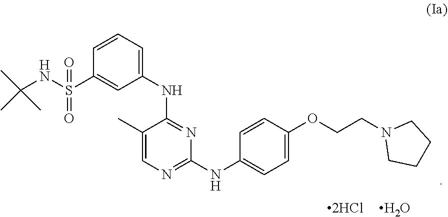 [SMILES: Cc1cnc(Nc2ccc(OCCN3CCCC3)cc2)nc1Nc1cccc(S(=O)(=O)NC(C)(C)C)c1 ; O]

25. The method of any one of claims 1 to 24, wherein the myeloproliferative disorder is myelofibrosis.

26. The method of claim 25, wherein the myelofibrosis is primary myelofibrosis.

27. The method of claim 26, wherein the primary myelofibrosis is selected from intermediate risk primary myelofibrosis and high risk primary myelofibrosis.

28. The method of claim 25, wherein the myelofibrosis is secondary myelofibrosis.

29. The method of claim 25, wherein the myelofibrosis is post-essential thrombocythemia myelofibrosis.

30. The method of claim 25, wherein the myelofibrosis is post-polycythemia vera myelofibrosis.

31. The method of any one of claims 1 to 24, wherein the myeloproliferative disorder is acute myeloid leukemia (AML).

32. The method of any one of claims 1 to 24, wherein the myeloproliferative disorder is polycythemia vera.

33. The method of any one of claims 1 to 24, wherein the myeloproliferative disorder is essential thrombocythemia.

34. The method of any one of claims 1 to 33, further comprising administering thiamine or a thiamine equivalent to the patient prior to, simultaneously with, or after the compound of formula (I), or a pharmaceutically acceptable salt and/or solvate thereof.

35. The method of claim 34, wherein the thiamine or thiamine equivalent is administered orally.

36. The method of claim 35, wherein the thiamine or thiamine equivalent is administered intravenously.

37. The method of any one of claims 1 to 36, further comprising administering a 5-HT3 receptor antagonist to the patient prior to, simultaneously with, or after the compound of formula (I), or a pharmaceutically acceptable salt and/or solvate thereof.

38. The method of claim 37, wherein the 5-HT3 receptor antagonist is ondasetron.

39. A method of increasing the safety and/or tolerability of a compound of formula (I): 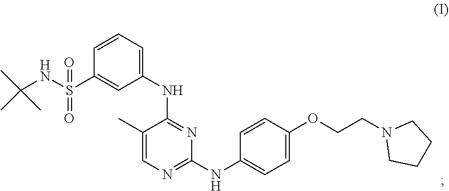 [SMILES: Cc1cnc(Nc2ccc(OCCN3CCCC3)cc2)nc1Nc1cccc(S(=O)(=O)NC(C)(C)C)c1]
or a pharmaceutically acceptable salt and/or a solvate thereof, the method comprising:
(a) administering to a patient concurrently receiving a dual CYP2C19 and CYP3A4 inhibitor a dose of a compound of formula (I) or a pharmaceutically acceptable salt and/or solvate thereof;
(b) monitoring the patient for adverse reactions; and
(c) in response to said monitoring, either maintaining or adjusting the dose of the dual CYP2C19 and CYP3A4 inhibitor in the patient to obtain a desired clinical effect.

40. The method of claim 39, wherein the dose of the compound of formula (I) or the pharmaceutically acceptable salt and/or solvate thereof is maintained.

41. The method of claim 39, wherein the dose of the compound of formula (I) or the pharmaceutically acceptable salt and/or solvate thereof is adjusted.

42. The method of claim 41, wherein the dose of the compound of formula (I) or the pharmaceutically acceptable salt and/or solvate thereof is lowered.

43. The method of any one of claims 39 to 42, wherein the solvate is a hydrate.

44. The method of any one of claims 39 to 43, wherein the pharmaceutically acceptable salt is a hydrochloride salt or a hydrate thereof.

45. The method of any one of claims 39 to 44, wherein the dihydrochloride monohydrate of the compound of formula (I) is administered.

46. The method of any one of claims 39 to 45, wherein the compound of formula (I), or the pharmaceutically acceptable salt and/or solvate thereof, is administered at a standard dose.

47. The method of claim 46, wherein the standard dose is about 400 mg/day.

48. The method of any one of claim 39 to 45 or 47, wherein the compound of formula (I), or the pharmaceutically acceptable salt and/or solvate thereof, is administered at a dose that is about ½ of a standard dose.

49. The method of any one of claim 39 to 45, 47, or 48, wherein the compound of formula (I), or the pharmaceutically acceptable salt and/or solvate thereof, is administered at a dose of about 200 mg/day.

50. The method of any one of claim 39 to 45 or 47, wherein the compound of formula (I), or the pharmaceutically acceptable salt and/or solvate thereof, is administered at a dose that is about ¼ of a standard dose.

51. The method of any one of claim 39 to 45, 47, or 50, wherein the compound of formula (I), or the pharmaceutically acceptable salt and/or solvate thereof, is administered at a dose of about 100 mg/day.

52. The method of any one of claims 39 to 51, wherein the compound of formula (I), or the pharmaceutically acceptable salt and/or solvate thereof, is administered once daily.

53. The method of any one of claim 39 to 45, or 47, wherein the compound of formula (I), or the pharmaceutically acceptable salt and/or solvate thereof, is administered at a dose that is about ⅛ a standard dose.

54. The method of any one of claim 39 to 45 or 53, wherein the compound of formula (I), or the pharmaceutically acceptable salt and/or solvate thereof, is administered at a dose of about 50 mg/day.

55. The method of any one of claims 39 to 54, wherein the compound of formula (I), or the pharmaceutically acceptable salt and/or solvate thereof, is administered every other day.

56. The method of any one of claim 39 to 45 or 53, wherein the compound of formula (I), or the pharmaceutically acceptable salt and/or solvate thereof, is administered at a dose of about 100 mg every other day.

57. The method of any one of claims 39 to 56, further comprising administering an increased dose of the compound of formula (I), or the pharmaceutically acceptable salt and/or solvate thereof, if the patient discontinues receiving the dual CYP2C19 and CYP3A4 inhibitor.

58. The method of claim 57, wherein the increased dose is less than the standard dose.

59. The method of claim 57, wherein the increased dose is the standard dose.

60. The method of any one of claims 39 to 59, wherein the safety and/or tolerability is increased relative to that when the standard dose is administered to the patient.

61. The method of any one of claims 39 to 60, wherein administering the compound of formula (I), or the pharmaceutically acceptable salt and/or solvate thereof, treats a myeloproliferative disorder in the patient and reduces one or more conditions selected from anemia, thrombocytopenia, gastrointestinal toxicity, hepatic toxicity, amylase elevation, and lipase elevation.

62. The method of any one of claims 39 to 61, wherein the increase in safety and/or tolerability comprises a reduction in toxicity and/or side effects.

63. The method of claim any one of claims 39 to 62, wherein the increased safety and/or tolerability comprises a reduction in one or more conditions selected from anemia, thrombocytopenia, gastrointestinal toxicity, hepatic toxicity, amylase elevation, and lipase elevation.

64. The method of claim 61, wherein the myeloproliferative disorder is myelofibrosis.

65. The method of claim 64, wherein the myelofibrosis is primary myelofibrosis.

66. The method of claim 65, wherein the primary myelofibrosis is selected from intermediate risk primary myelofibrosis and high risk primary myelofibrosis.

67. The method of claim 64, wherein the myelofibrosis is secondary myelofibrosis.

68. The method of claim 64, wherein the myelofibrosis is post-essential thrombocythemia myelofibrosis.

69. The method of claim 64, wherein the myelofibrosis is post-polycythemia vera myelofibrosis.

70. The method of claim 61, wherein the myeloproliferative disorder is acute myeloid leukemia (AML).

71. The method of claim 61, wherein the myeloproliferative disorder is polycythemia vera.

72. The method of claim 61, wherein the myeloproliferative disorder is essential thrombocythemia.

73. The method of any one of claims 39 to 72, wherein the compound of formula (I), or the pharmaceutically acceptable salt and/or solvate thereof, is administered orally.

74. The method of any one of claims 39 to 73, wherein the dual CYP2C19 and CYP3A4 inhibitor is selected from itraconazole, fluconazole, fluvoxamine, voriconazole, and combinations thereof.

75. The method of any one of claims 39 to 74, wherein the dual CYP2C19 and CYP3A4 inhibitor is fluconazole.

76. The method of any one of claims 39 to 75, wherein the patient is administered a compound of formula (Ia):

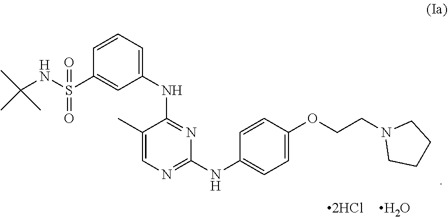 [SMILES: Cc1cnc(Nc2ccc(OCCN3CCCC3)cc2)nc1Nc1cccc(S(=O)(=O)NC(C)(C)C)c1 ; O]

77. The method of any one of claims 39 to 76, further comprising administering thiamine or a thiamine equivalent to the patient prior to, simultaneously with, or after the compound of formula (I), or a pharmaceutically acceptable salt and/or solvate thereof.

78. The method of claim 77, wherein the thiamine or thiamine equivalent is administered orally.

79. The method of claim 77, wherein the thiamine or thiamine equivalent is administered intravenously.

80. The method of any one of claims 39 to 79, further comprising administering a 5-HT3 receptor antagonist to the patient prior to, simultaneously with, or after the compound of formula (I), or a pharmaceutically acceptable salt and/or solvate thereof.

81. The method of claim 80, wherein the 5-HT3 receptor antagonist is ondasetron.
